(19) United States
(12) Patent Application Publication
(10) Pub. No.: US 2026/0188435 A1
(43) Pub. Date: Jul. 2, 2026

(54) METHOD FOR SELECTING COLLECTOR FOR SYNCHRONOUS LEAD-SILVER ENRICHMENT BASED ON COORDINATION CHEMISTRY AND ELECTROCHEMISTRY

(71) Applicant: Central South University, Changsha (CN)

(72) Inventors: Chenyang ZHANG, Changsha (CN); Hongliang ZHANG, Changsha (CN); Wei SUN, Changsha (CN); Heng YU, Changsha (CN); Xin RAO, Changsha (CN); Minsheng FU, Changsha (CN)

(73) Assignee: Central South University, Changsha (CN)

(21) Appl. No.: 19/392,218

(22) Filed: Nov. 18, 2025

(30) Foreign Application Priority Data
Dec. 31, 2024 (CN) 2024119854089

(51) Int. Cl. G16C 10/00; G01N 27/27; G01N 33/2028

(52) U.S. Cl. CPC G16C 10/00; G01N 27/27; G01N 33/2028

(57) ABSTRACT

A method for selecting a collector for synchronous lead-silver enrichment based on coordination chemistry and electrochemistry includes: acquiring electrochemical information on a solid-solid interaction between a silver mineral and galena; acquiring the galena, the silver mineral, and potential targeted mineral-philic groups of the galena and the silver mineral, and outputting chk files; analyzing the quantum chemical calculation chk files to obtain shape and energy level information of frontier molecular orbitals of related minerals and the mineral-philic groups; preliminarily identifying a targeted mineral-philic group for the silver mineral; thermodynamically, determining whether the targeted mineral-philic group preliminarily identified for the silver mineral demonstrates a better adsorption capacity than other mineral-philic groups for the silver mineral; and calculating solvation free energies of targeted mineral-philic group derivatives with different carbon chain lengths and shapes, and determining a final mineral-philic reagent.

### CROSS REFERENCE TO THE RELATED APPLICATIONS

This application is based upon and claims priority to Chinese Patent Application No. 202411985408.9, filed on Dec. 31, 2024, the entire contents of which are incorporated herein by reference.

### TECHNICAL FIELD

The present disclosure relates to mineral flotation, and specifically relates to a method for selecting a collector for synchronous lead-silver enrichment based on coordination chemistry and electrochemistry.

### BACKGROUND

Silver often occurs as an associated element in various types of ores, and typically coexists with other metals. Silver rarely forms independent deposits (with only a few exceptions) and is primarily associated with lead-zinc ores. Normally, during lead-zinc ore beneficiation, silver can be enriched to a grade of 2,000 g/t or higher in a lead concentrate, and to a grade of about 200 g/t in a zinc concentrate. Due to the similar properties of zinc and silver, the metallurgical separation of zinc and silver is difficult. Consequently, the pricing coefficient for silver in zinc concentrates is relatively low (only 0.2), and the silver grade must be no less than 200 g/t. In contrast, the subsequent metallurgical separation of lead and silver is straightforward, and thus the pricing coefficient for silver in lead concentrates is higher, reaching 0.85. To maximize the value of associated silver, it is necessary to enrich as much of the associated silver as possible into a lead concentrate.

Interactions of collectors with mineral surfaces include physical adsorption, chemical adsorption, and surface chemical reactions. The adsorption of collectors is closely related to the flotation behaviors of minerals. Within a specified concentration range of a collector, as the concentration of the collector increases, the adsorption capacity increases, leading to a significantly improved flotation recovery. When the concentration of the collector reaches a given value, the increase of the flotation recovery with the concentration and adsorption capacity enhancement slows down. When the concentration of the collector is excessively high, the adsorption capacity may continue to grow, but the flotation recovery no longer increases, and may even decline. Therefore, during a flotation process, it is essential to identify an appropriate collector and accurately control a dosage of the collector to achieve the optimal efficiency.

The previous flotation recovery of associated silver minerals from lead-zinc ores typically relies on the “trial-and-error” method for reagent screening. However, reagents selected by the “trial-and-error” method often have poor accuracy, and exhibit low enhanced recovery efficiency for associated silver minerals in lead-zinc ores.

### SUMMARY

To address the aforementioned problem that reagents selected by the “trial-and-error” method often have poor accuracy and exhibit low enhanced recovery efficiency for associated silver minerals in lead-zinc ores, the present disclosure provides a method for selecting a collector for synchronous lead-silver enrichment based on coordination chemistry and electrochemistry. This method can select and design a targeted flotation collector favorable for the synchronous enrichment and recovery of lead and silver.

To achieve the above objective, an aspect of the present disclosure provides a method for selecting a collector for synchronous lead-silver enrichment based on coordination chemistry and electrochemistry, including the following steps:
S1, based on a quantum chemical calculation and a specific flotation environment, establishing a mineral solid-solid interaction model to acquire electrochemical information on a solid-solid interaction between a silver mineral and galena;
S2, conducting first-principles quantum chemical calculations to acquire the galena, the silver mineral, and potential targeted mineral-philic groups of the galena and the silver mineral, and outputting chk files;
S3, analyzing the quantum chemical calculation chk files to acquire shape and energy level information of frontier molecular orbitals of related minerals and the mineral-philic groups;
S4, based on three principles of bonding, screening the mineral-philic groups through coordination chemistry properties of frontier molecular orbitals of the minerals with frontier molecular orbitals of the targeted mineral-philic groups to preliminarily identify a targeted mineral-philic group for the silver mineral;
S5, thermodynamically, determining whether the targeted mineral-philic group preliminarily identified for the silver mineral demonstrates a better adsorption capacity than other mineral-philic groups for the silver mineral; and if so, proceeding to S6; and
S6, calculating solvation free energies of targeted mineral-philic group derivatives with different carbon chain lengths and shapes according to energy information in log files resulting from quantum chemical calculations, and selecting a mineral-philic group derivative with a minimum solvation free energy as a final mineral-philic reagent.

Specifically, in the S1, based on the mineral solid-solid interaction model, density functional theory (DFT) calculations are conducted at a level not lower than a generalized gradient approximation-Perdew-Wang 91 (GGA-PW91) functional to acquire the electrochemical information on the solid-solid interaction between the silver mineral and the galena.

More specifically, the electrochemical information on the solid-solid interaction between the silver mineral and the galena is as follows: during an electrochemical process, the galena with a low potential undergoes an anodic reaction to lose an electron and thus is positively charged, while the silver mineral with a high potential undergoes a cathodic reaction to accept an electron and thus is negatively charged.

Preferably, in the S2, with effects of dispersion and solvation of minerals and mineral-philic groups fully considered, the first-principles quantum chemical calculations are conducted at a level not lower than a Becke, 3-parameter, Lee-Yang-Parr (B3LYP) functional and a DEF2TZVP basis set to acquire the galena, the silver mineral, and the potential targeted mineral-philic groups of the galena and the silver mineral.

More preferably, the potential targeted mineral-philic groups include monothiocarbonate, xanthate, trithiocarbonate, monothiophosphate, dithiophosphate, thiosulfate, thiosulfonate, dithiocarbamate, mercaptan, thionocarbamate, thiourea, or thioamide.

Specifically, in the S3, the frontier molecular orbitals of the related minerals and the mineral-philic groups include highest occupied molecular orbitals (HOMOs) and lowest unoccupied molecular orbitals (LUMOs).

Further, in the S4, LUMOs of the targeted mineral-philic group preliminarily identified for the silver mineral are capable of symmetrically matching HOMOs of argentite and elemental silver.

Further, a monothiophosphate (dithiophosphate) and a thiosulfonate (thiosulfate) that have a pₓ orbital as LUMOs are preliminarily determined as the targeted mineral-philic group for the silver mineral.

Further, in the S6, the final mineral-philic reagent is diisobutyl dithiophosphate.

Through the above technical solutions, the present disclosure achieves the following beneficial effects:
1. In the present disclosure, based on the unique electrochemical properties of a solid-solid interaction between sulfide minerals and the coordination chemistry mechanisms (conventional orbital bonding interaction and orbital backbonding interaction) of common mineral-philic groups with mineral surfaces, targeted mineral-philic groups for the synchronous recovery of lead and silver are selected. Hydrophobic groups are designed for the selected targeted mineral-philic groups based on solvation free energies. Finally, a targeted flotation collector conducive to the synchronous enrichment and recovery of lead and silver is selected and designed.
2. The present disclosure combines the electrochemistry of sulfide mineral flotation with the coordination chemistry of flotation for the first time, and overcomes the limitations of previous reagent screening designs. Consequently, the present disclosure provides a novel method for the research, development, and design of highly-effective collectors for associated and coexisting rare metal resources.

### BRIEF DESCRIPTION OF THE DRAWINGS

FIG. 1 is a technical route for the selection and design of a targeted collector for the synchronous recovery of lead and silver in Example 1 of the present disclosure;

FIG. 2 shows a solid-solid interaction model between a silver mineral and galena;

FIG. 3 is a schematic diagram of an electrochemical behavior of a solid-solid interaction between a silver mineral and galena;

FIGS. 4A-4C show interfacial coordination mechanisms of a targeted collector diisobutyl monothiophosphate (DIBMTP) with galena and silver minerals, where FIG. 4A shows a conventional orbital bonding interaction between DIBMTP and galena, FIG. 4B shows a orbital backbonding interaction between DIBMTP and argentite as a typical silver mineral, and FIG. 4C shows a orbital backbonding interaction between DIBMTP and elemental silver as a typical silver mineral; and

FIG. 5 is a closed-circuit flotation flowsheet for lead (silver) in Example 2 of the present disclosure.

### DETAILED DESCRIPTION OF THE EMBODIMENTS

The specific implementations of the present disclosure will be described in detail below with reference to embodiments. It should be understood that the specific implementations described herein are merely intended to illustrate and explain the present disclosure, rather than to limit the present disclosure.

### Example 1 Selection of a Collector for Synchronous Lead-Silver Enrichment Based on Coordination Chemistry and Electrochemistry

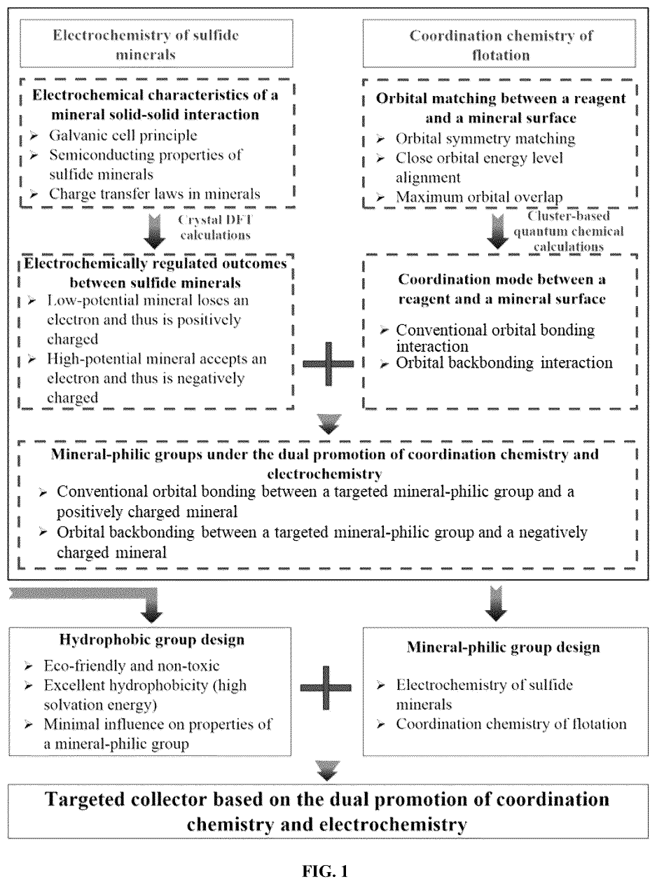

A technical route for the selection of a collector for synchronous lead-silver enrichment based on coordination chemistry and electrochemistry is shown in FIG. 1.

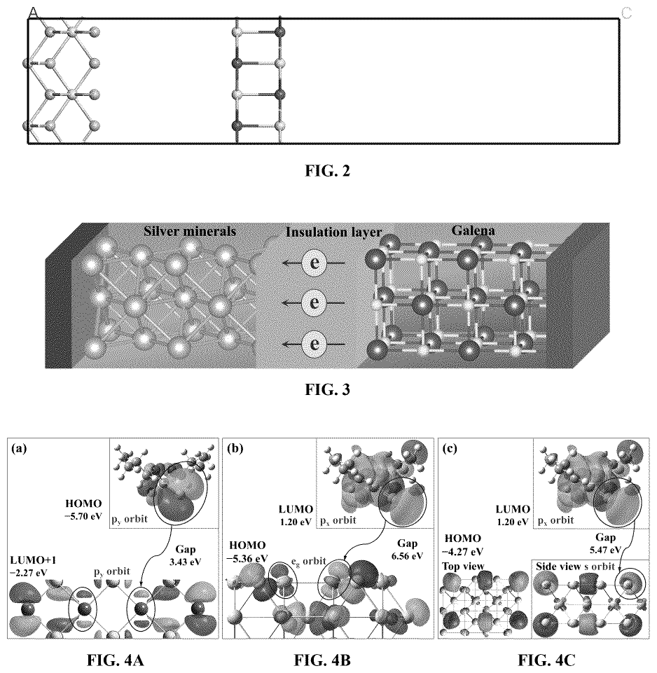

S1. Since mineral flotation is conducted in an aqueous solution, mineral particles in a slurry will inevitably undergo solid-solid interactions during grinding, stirring, and flotation processes. Thus, based on periodic quantum chemical calculations and considering mutual collisions of mineral particles in a specific flotation environment, a mineral solid-solid interaction model is established, as shown in FIG. 2. Based on the mineral solid-solid interaction model, crystal DFT calculations are conducted at a level not lower than a GGA-PW91 functional to acquire electrochemical information on a solid-solid interaction between a silver mineral and galena. It is determined that, during an electrochemical process of the solid-solid interaction between the silver mineral and the galena, the galena with a low potential (Pb²⁺→Pb⁰ −0.125 V and S⁰→S²⁻ −0.447 V) undergoes an anodic reaction ([PbS]→[Pbs]ⁿ⁺+n·e) to lose an electron and thus is positively charged, while argentite as a typical silver mineral with a high potential (Ag⁺→Ag⁰ 0.799 V) undergoes a cathodic reaction ([AgS]+n·e→[AgS]ⁿ⁻) to accept an electron and thus is negatively charged, as shown in FIG. 3.

S2. With effects of dispersion and solvation of minerals and mineral-philic groups fully considered, first-principles quantum chemical calculations are conducted at a level not lower than a B3LYP functional and a DEF2TZVP basis set to acquire chk files of the galena, the silver mineral, and potential targeted mineral-philic groups of the galena and the silver mineral (monothiocarbonate, xanthate, trithiocarbonate, monothiophosphate, dithiophosphate, thiosulfate, thiosulfonate, dithiocarbamate, mercaptan, thionocarbamate, thiourea, and thioamide groups). The chk files are output.

S3. The quantum chemical calculation (cluster-based quantum chemical calculations) output files (chk files) are analyzed to acquire shape and energy level information of frontier molecular orbitals (including HOMOs and LUMOs) of related minerals and the mineral-philic groups.

S4. Based on three principles of bonding, namely, orbital symmetry matching, maximum orbital overlap, and close orbital energy level alignment, it is determined that monothiophosphate (dithiophosphate) and thiosulfonate (thiosulfate) groups that have a pₓ orbital as LUMOs can symmetrically match HOMOs of argentite (e_{g} orbital) and elemental silver (s orbital). As a result, these groups are preliminarily identified as targeted mineral-philic groups for the silver minerals. In contrast, LUMOs of other mineral-philic groups are p_{z} orbitals, which do not match the HOMOs of the silver minerals. As a result, these mineral-philic groups are unsuitable for the enhanced collection of the silver minerals. Based on coordination chemistry properties between the minerals and the targeted mineral-philic groups, a mineral-philic reagent is selected.

S5. Based on the targeted mineral-philic groups preliminarily identified, namely, monothiophosphate, dithiophosphate, thiosulfonate, and thiosulfate groups, interfacial adsorption simulations are conducted. Whether the monothiophosphate group demonstrates a better adsorption capacity than other mineral-philic groups for the silver mineral is determined thermodynamically. If so, the monothiophosphate group should be regarded as a targeted mineral-philic group for the synchronous recovery of lead and silver.

S6. Solvation free energies of monothiophosphate derivatives with different carbon chain lengths and shapes are calculated according to energy information in log files resulting from quantum chemical calculations. A monothiophosphate derivative with a minimum solvation free energy is selected as a final mineral-philic reagent. The final mineral-philic reagent is DIBMTP.

The targeted collector DIBMTP for synchronous lead-silver enrichment selected based on coordination chemistry and electrochemistry in the present disclosure exhibits different coordination chemistry properties for silver minerals and galena. The key of the targeted collector to achieving the synchronous recovery of lead and silver lies in the distinct surface coordination bonding mechanisms: A coordination interaction of the targeted collector with galena is a conventional orbital bonding interaction, which involves the HOMO (p_{y} orbital) of DIBMTP and the LUMO+1 orbital of galena. A coordination interaction of the targeted collector with a silver mineral is a orbital backbonding interaction, which involves the pₓ orbital as LUMOs of DIBMTP and the s and e_{g} orbitals as HOMOs of elemental silver and argentite, as shown in FIGS. 4A-4C. Based on the electrochemical properties of a solid-solid interaction between a silver mineral and galena and the coordination chemistry properties of these minerals with a flotation reagent, it can be determined that an electrochemical interaction between the silver mineral and the galena promotes the electron transfer from the galena to the silver mineral, and thus the galena is electron-deficient and possesses enhanced positive charges, which facilitates the conventional orbital bonding interaction with DIBMTP. Moreover, the silver mineral exhibits an enhanced electron-donating capability after receiving electrons, which promotes the orbital backbonding interaction with DIBMTP. The collector DIBMTP designed by this method demonstrates outstanding performance in a practical flotation experiment, and enables a significantly improved recovery of silver in a lead concentrate than the traditional collectors.

### Example 2 Validation of a Synchronous Enrichment Effect of DIBMTP for Lead and Silver in a Silver-Containing Lead-Zinc Ore

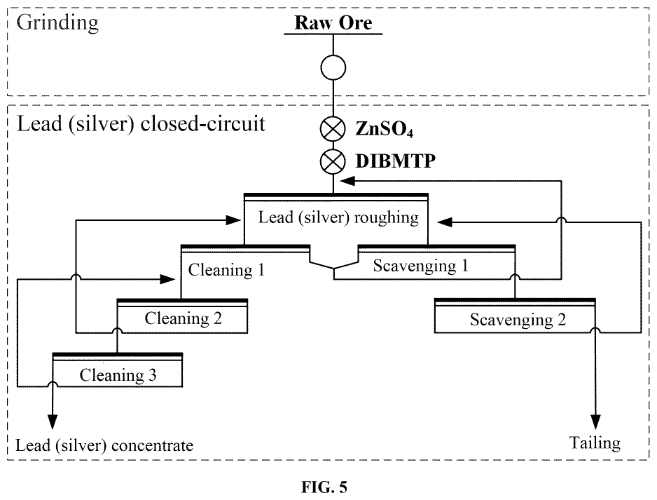

A flotation process for an exemplary concentrate in a Pb (Ag) closed-circuit flotation flowsheet is shown in FIG. 5: 500 g of a raw ore with a particle size of 2 mm was ground in a ball mill to an appropriate fineness, and then transferred to a flotation cell. Roughing and scavenging were conducted in a 1.5 L flotation cell. First cleaning was conducted in a 1.0 L flotation cell, and second and third cleaning were conducted in a 0.5 L flotation cell. During a flotation test, a depressant ZnSO₄ and a collector DIBMTP were added in desired amounts to a flotation cell, with a conditioning time of 3 min for each reagent. During a flotation process, a mineral was separated at a natural pH (approximately 7.0) without any pH adjusting agent.

Additionally, a comparative test was conducted with diethyl dithiocarbamate (DDTC) or diisobutyl dithiophosphate (DIBDTP) instead of DIBMTP.

Results are shown in Table 1.

TABLE 1
Indexes of various products in lead (silver) closed-circuit flotation experiments

| Reagent | | Grade (%) | | | Recovery (%) | | |
|---|---|---|---|---|---|---|---|
| system | Product | Pb | Zn | Ag (g/t) | Pb | Zn | Ag |
| DDTC | Concentrate | 7.71 | 2.19 | 216 | 14.49 | 1.82 | 18.73 |
| | Tailing | 2.97 | 7.76 | 61 | 85.51 | 98.18 | 81.27 |
| | Raw ore | 3.26 | 7.42 | 70.80 | 100.0 | 100.0 | 100.0 |
| DIBDTP | Concentrate | 60.67 | 2.26 | 629 | 90.58 | 1.47 | 42.03 |
| | Tailing | 0.32 | 7.70 | 44 | 9.42 | 98.53 | 57.97 |
| | Raw ore | 3.23 | 7.43 | 72.24 | 100.0 | 100.0 | 100.0 |
| DIBMTP | Concentrate | 59.02 | 2.59 | 668 | 90.63 | 1.73 | 46.27 |
| | Tailing | 0.32 | 7.71 | 41 | 9.37 | 98.27 | 53.73 |
| | Raw ore | 3.24 | 7.45 | 71.91 | 100.0 | 100.0 | 100.0 |

According to the results, DDTC suitable for high-soda lime systems cannot concentrate lead and silver minerals in a near-neutral alkali-free medium. Grades of lead and silver in a corresponding concentrate are only 7.71% and 216 g/t, respectively, and recoveries of lead and silver are 14.49% and 18.73%, respectively, which do not meet the smelting standard (a lead content should be no less than 30%). When DIBDTP is adopted as a collector, a lead concentrate product with lead and silver grades of 60.67% and 629 g/t, respectively, can be obtained, and recoveries of lead and silver are 90.58% and 42.03%, respectively. Compared to the use of DIBDTP as a collector, the use of DIBMTP as a collector can significantly improve the recovery of silver in a concentrate to 46.27%. With comparable lead recoveries, the use of DIBMTP as a collector remarkably increases the silver recoveries by approximately 4.2% compared with the use of DIBDTP as a collector, indicating that DIBMTP is a high-performance silver-friendly collector.

Preferred embodiments of the present disclosure are described in detail above, but the present disclosure is not limited to specific details in the above embodiments. Various simple variations can be made to the technical solutions of the present disclosure without departing from the technical ideas of the present disclosure, and these simple variations fall within the protection scope of the present disclosure.

In addition, it should be noted that various specific technical features described in the above specific embodiments can be combined in any suitable manner, provided that there is no contradiction. To avoid unnecessary repetition, various possible combination modes of the present disclosure are not described separately.

The different embodiments of the present disclosure can also be combined arbitrarily, as long as such combinations do not deviate from the spirit of the present disclosure. These combinations shall also be deemed part of the content disclosed in the present disclosure.

What is claimed is:

1. A method for selecting a collector for synchronous lead-silver enrichment based on coordination chemistry and electrochemistry, comprising following steps:
S1, based on a quantum chemical calculation and a specific flotation environment, establishing a mineral solid-solid interaction model; and based on the mineral solid-solid interaction model, conducting density functional theory (DFT) calculations at a level not lower than a generalized gradient approximation-Perdew-Wang 91 (GGA-PW91) functional to acquire electrochemical information on a solid-solid interaction between a silver mineral and galena, wherein the electrochemical information on the solid-solid interaction between the silver mineral and the galena is as follows: during an electrochemical process, the galena with a low potential undergoes an anodic reaction to lose an electron and thus is positively charged, while the silver mineral with a high potential undergoes a cathodic reaction to accept an electron and thus is negatively charged;
S2, while fully considering effects of dispersion and solvation of minerals and mineral-philic groups, conducting first-principles quantum chemical calculations at a level not lower than a Becke, 3-parameter, Lee-Yang-Parr (B3LYP) functional and a DEF2TZVP basis set to acquire the galena, the silver mineral, and potential targeted mineral-philic groups of the galena and the silver mineral, and outputting chk files, wherein the potential targeted mineral-philic groups comprise monothiocarbonate, xanthate, trithiocarbonate, monothiophosphate, dithiophosphate, thiosulfate, thiosulfonate, dithiocarbamate, mercaptan, thionocarbamate, thiourea, or thioamide;
S3, analyzing the quantum chemical calculation chk files to acquire shape and energy level information of frontier molecular orbitals of related minerals and the mineral-philic groups;
S4, based on three principles of bonding, screening the mineral-philic groups through coordination chemistry properties of frontier molecular orbitals of the minerals with frontier molecular orbitals of the targeted mineral-philic groups to preliminarily identify a targeted mineral-philic group for the silver mineral;
S5, thermodynamically determining whether the targeted mineral-philic group preliminarily identified for the silver mineral demonstrates a better adsorption capacity than other mineral-philic groups for the silver mineral; and if the targeted mineral-philic group preliminarily identified for the silver mineral demonstrates a better adsorption capacity than other mineral-philic groups for the silver mineral, proceeding to S6; and
S6, calculating solvation free energies of targeted mineral-philic group derivatives with different carbon chain lengths and shapes according to energy information in log files resulting from quantum chemical calculations, and selecting a mineral-philic group derivative with a minimum solvation free energy as a final mineral-philic reagent.

2. The method according to claim 1, wherein in the S3, the frontier molecular orbitals of the related minerals and the mineral-philic groups comprise highest occupied molecular orbitals (HOMOs) and lowest unoccupied molecular orbitals (LUMOs).

3. The method according to claim 2, wherein in the S4, LUMOs of the targeted mineral-philic group preliminarily identified for the silver mineral are capable of symmetrically matching HOMOs of argentite and elemental silver.

4. The method according to claim 3, wherein a monothiophosphate or a dithiophosphate and a thiosulfonate or a thiosulfate that have a pₓ orbital as LUMOs are preliminarily determined as the targeted mineral-philic group for the silver mineral.

5. The method according to claim 4, wherein in the S6, the final mineral-philic reagent is diisobutyl dithiophosphate.
